(19) United States
(12) Patent Application Publication
(10) Pub. No.: US 2020/0168312 A1
(43) Pub. Date: May 28, 2020

(54) AUTONOMICALLY DRIVEN ARTIFICIAL INTELLIGENCE MEDICAL SUCTION DEVICE TO ALLEVIATE PAIN OF PATIENT AND METHOD OF CONTROLLING ARTIFICIAL INTELLIGENCE MEDICAL SUCTION DEVICE

(71) Applicant: LMECA CO., LTD., Gangwon-do (KR)

(72) Inventor: Jung-kil KANG, Gyeonggi-do (KR)

(21) Appl. No.: 16/089,907

(22) PCT Filed: Jul. 14, 2016
(86) PCT No.: PCT/KR2016/007668

§ 371 Date: Sep. 28, 2018

(30) Foreign Application Priority Data
Mar. 29, 2016 (KR) 10-2016-0037433

(51) Int. Cl. G16H 20/40; A61M 1/00

(52) U.S. Cl. CPC G16H 20/40; A61M 1/0031; A61M 1/008; A61M 2205/3375; A61M 25/007; A61M 2230/205; A61M 2210/1039; A61M 2205/3334; A61M 2230/04

(57) ABSTRACT

A control of an artificial intelligence medical suction device may be embodied by a controller through the steps of: setting a suction pressure of a suction pump connected to the catheter to be a predetermined reference suction pressure, moving the catheter forward so that a suction end portion of the catheter is inserted into the respiratory organ, and analyzing a state of the suction end portion of the catheter based on a variation value of an actual suction pressure measured from the catheter. The medical suction device is capable of determining whether the suction end portion thereof is adsorbed to an inner wall of bronchial tubes on its own.

### TECHNICAL FIELD

The present invention relates to an artificial intelligence medical suction device, and a method of controlling the artificial intelligence medical suction device, and more specifically, to an autonomically driven artificial intelligence medical suction device to alleviate pain of a patient, which is capable of determining whether a catheter is adsorbed to an inner wall of bronchial tubes by the medical suction device on its own, so that an occurrence of a situation, in which the pain of the patient is increased due to a suction end portion of the catheter is absorbed to the inner wall of the bronchial tubes, can be prevented, and a method of controlling the artificial intelligence medical suction device.

### BACKGROUND ART

A medical suction device is a medical foreign material suction device which sucks in and removes by force into a container foreign materials such as blood, saliva, vomitus and secreta that are generated from an inside of patient's body while operating on the patient in hospitals.

In general, patients with impaired mobility at home or hospital have a suction device mounted constantly for a guardian or nurse to drain foreign material out of the trachea or bronchial tubes.

However, since foreign materials may be generated during sleep to block the trachea, the nurse, carer or guardian should operate the suction device from time to time. In addition, these guardians have to constantly check the condition of the patient at all times, as well as have the difficulty of removing foreign matters by driving the suction device from time to time.

In addition, when inserting the catheter provided in the medical suction device into a respiratory organ of the patient after determining that the foreign matters such as sputum exist in the respiratory organ of the patient, if an insertion direction of the catheter is inaccurate, a situation, in which a suction end portion of the catheter is absorbed to an inner wall of bronchial tubes, may occur. In this case, there is a problem that the patient feels severe pain.

### SUMMARY OF INVENTION

### Problems to be Solved by Invention

Accordingly, it is an object of the present invention to provide an artificial intelligence medical suction device which is capable of determining whether a catheter is adsorbed to an inner wall of bronchial tubes by the medical suction device on its own, so that an occurrence of a situation, in which the pain of the patient is increased due to a suction end portion of the catheter is absorbed to the inner wall of the bronchial tubes, can be prevented, and a method of controlling the artificial intelligence medical suction device.

### Means for Solving Problems

To achieve the above object, in a method of controlling an artificial intelligence medical suction device which removes foreign matters in a respiratory organ using a catheter, the method of controlling an artificial intelligence medical suction device according to an aspect of the present invention includes: (a) setting, by a controller, a suction pressure of a suction pump connected to the catheter to be a predetermined reference suction pressure; (b) moving, by the controller, the catheter forward so that a suction end portion of the catheter is inserted into the respiratory organ; and (c) analyzing, by the controller, a state of the suction end portion of the catheter based on a variation value of an actual suction pressure measured inside of the catheter.

Preferably, in step (c), when the catheter is maintained with the actual suction pressure measured therein being increased by a predetermined ratio or more for a predetermined time or longer, the controller determines that the suction end portion of the catheter is adsorbed to an inner wall of bronchial tubes.

Meanwhile, an artificial intelligence medical suction device according to another aspect of the present invention includes: a suction pump connected to the catheter; and a controller configured to set a suction pressure of the suction pump to be a predetermined reference suction pressure, move the catheter forward so that a suction end of the catheter is inserted into a respiratory organ, and analyze a state of a suction end portion of the catheter based on a variation value of an actual suction pressure measured inside of the catheter, wherein, when the catheter is maintained with the actual suction pressure measured therefrom being increased by a predetermined ratio or more for a predetermined time or longer, the controller determines that the suction end portion of the catheter is adsorbed to an inner wall of bronchial tubes.

### Advantageous Effects

According to the present invention, since the medical suction device may determine whether the catheter is adsorbed to the inner wall of the bronchial tubes on its own, an occurrence of a situation, in which the pain of the patient is increased due to a suction end portion of the catheter is absorbed to the inner wall of the bronchial tubes, can be prevented.

### BRIEF DESCRIPTION OF THE DRAWINGS

FIG. 1 is a block diagram illustrating a structure of an artificial intelligence medical suction device according to an embodiment of the present invention.

FIG. 2 is a flowchart illustrating a procedure and steps for executing a method of controlling the artificial intelligence medical suction device according to the embodiment of the present invention.

FIG. 3 is a graph illustrating waveforms of auscultatory sounds measured from a patient without respiratory disturbance due to sputum or the like with being converted into a frequency domain.

FIG. 4 is a graph illustrating waveforms of auscultatory sounds measured from a patient suffering from the respiratory disturbance due to the sputum or the like with being converted into a frequency domain.

FIG. 5 is a flowchart illustrating a procedure and steps for executing a control method of an artificial intelligence medical suction device capable of determining whether a catheter is adsorbed to an inner wall of bronchial tubes according to another embodiment of the present invention.

FIG. 6 is a view illustrating a structure of a suction end portion of the catheter provided in the artificial intelligence medical suction device according to the present invention.

### MODE FOR CARRYING OUT INVENTION

Hereinafter, the present invention will be described in detail with reference to the accompanying drawings. Referring to the drawings, wherein like reference characters designate like or corresponding parts throughout the several views. In the embodiments of the present invention, the publicly known functions and configurations that are judged to be able to make the purport of the present invention unnecessarily obscure will not be described.

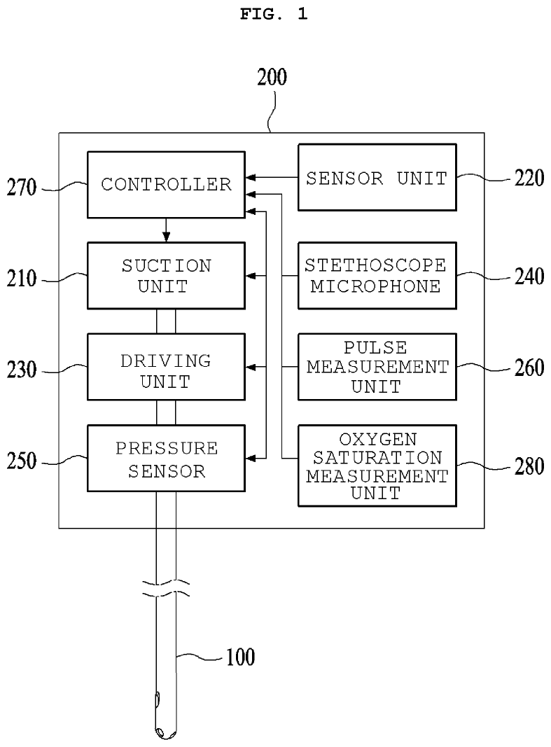

FIG. 1 is a block diagram illustrating a structure of an artificial intelligence medical suction device according to an embodiment of the present invention. An artificial intelligence medical suction device 200 according to the embodiment of the present invention performs a function of removing foreign matters in a respiratory organ of a patient using a catheter 100. To this end, the medical suction device includes a suction pump 210 installed by connecting to one end of the catheter 100, a sensor unit 220, a driving unit 230, a stethoscope microphone 240, a pressure sensor 250, a pulse measurement unit 260, a controller 270, and an oxygen saturation measurement unit 280.

First, the suction pump 210 is installed at one end of the catheter 100, and generates a suction pressure inside the catheter 100 so that foreign matters such as sputum are sucked through the other end of the catheter 100 with being inserted into the respiratory organ of the patient.

The sensor unit 220 includes a mass flow meter (MFM) sensor for measuring masses of inspiratory gas and exhalatory gas of the patient, respectively, and performs a function of measuring the masses of inspiratory gas and exhalatory gas of the patient and transmitting the measured results to the controller 270.

Meanwhile, the driving unit 230 moves the catheter 100 forward to insert the catheter 100 into bronchial tubes, or moves the catheter 100 backward to remove the catheter 100 from the bronchial tubes.

The stethoscope microphone 240 measures auscultatory sounds of the patient and transmits the measured auscultatory sounds to the controller 270. The pulse measurement unit 260 measures a pulse rate of the patient and transmits the measured pulse rate to the controller 270. The oxygen saturation measurement unit 280 measures an oxidation saturation from a blood sample collected from the patient and transmits the measured oxygen saturation to the controller 270.

Meanwhile, the controller 270 performs a function of determining whether it is necessary to remove foreign matters such as sputum in the bronchial tubes of the patient based on information on conditions of the patient including information on auscultatory sounds of the patient received from the stethoscope microphone 240, information on pulse rates of the patient received from the pulse measurement unit 260, and information on oxygen saturations of the patient received from the oxygen saturation measurement unit 280.

In addition, the pressure sensor 250 measures a pressure (negative pressure) formed inside the catheter 100, and transmits the measured pressure value to the controller 270.

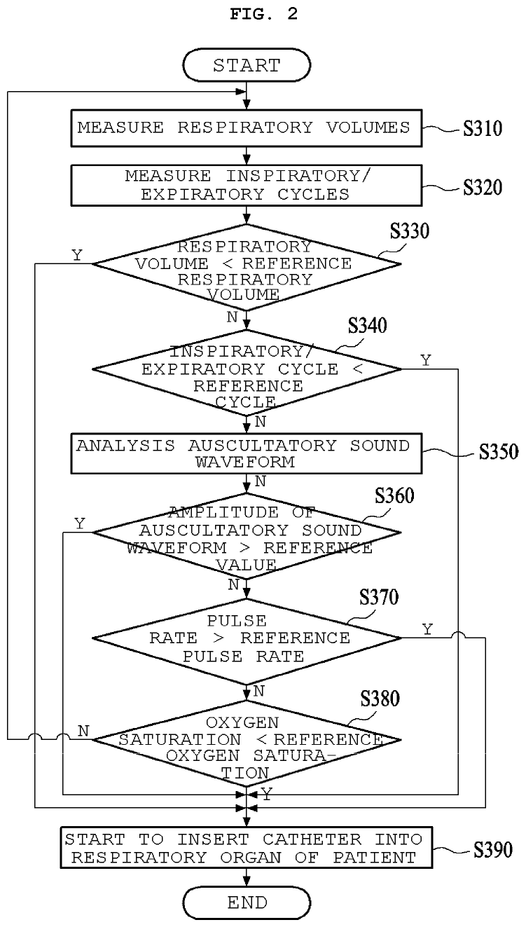

FIG. 2 is a flowchart illustrating a procedure and steps for executing a method of controlling the artificial intelligence medical suction device 200 according to the embodiment of the present invention. Hereinafter, the steps for executing the method of controlling the artificial intelligence medical suction device 200 according to the embodiment of the present invention will be described with reference to FIGS. 1 and 2.

First, the sensor unit 220 includes a first mass flow meter sensor and a second mass flow meter sensor. The first mass flow meter sensor measures an expiratory tidal volume of the patient from an expiratory outlet port of a respiratory mask worn by the patient and the second mass flow meter sensor measures an inspiratory tidal volume of the patient from an inspiratory inlet port of the respiratory mask.

Meanwhile, the controller 270 alternately receives the measured value of the expiratory tidal volume of the patient from the first mass flow meter sensor and the measured value of the inspiratory tidal volume of the patient from the second mass flow meter sensor. As a result, the controller 270 can secure and determine information on tidal volumes per respiratory cycle of the patient in real time based on the measured values received from the first and second mass flow meter sensors.

In addition, the controller 270 calculates an interval between the time when the measured value of the expiratory tidal volume is received from the first mass flow meter sensor and the time when the measured value of the next expiratory tidal volume is received, thereby measuring the respiratory (inspiratory/expiratory) cycle of the patient (S320).

As described above, the information on respiratory volumes of the patient and information on the respiratory cycles of the patient are accumulated and stored in the controller 270 in real time. The controller 270 calculates and stores a cumulative average value of the respiratory volumes of the patient based on such information, and calculates and stores a cumulative average value of the respiratory cycles of the patient.

Meanwhile, the controller 270 determines whether the tidal volumes per respiratory cycle of the patient received from the sensor unit 220 in real time is less than a cumulative average value of the respiratory volumes (‘reference respiratory volume’) of the patient (S330).

In general, when the foreign matters such as sputum are accumulated in the respiratory organ at a predetermined level or more, the patient's breathing becomes challenging. As a result, the respiratory cycle is shortened and the tidal volumes per respiratory cycle are reduced.

Accordingly, when it is determined by the controller 270 that the tidal volume per respiratory cycle of the patient is less than the cumulative average value of the respiratory volumes (reference respiratory volume) of the patient, the controller 270 determines that it is necessary to remove the sputum from the patient, and transmits operation start commands to the suction pump 210 and the driving unit 230. Thereby, the catheter 100 starts to be inserted into the respiratory organ of the patient (S390).

Meanwhile, in the above-described step S330, if it is determined that the tidal volume per respiratory cycle of the patient is the cumulative average value of the respiratory volumes (reference respiratory volume) or more of the patient, the controller 270 determines whether the current respiratory cycle of the patient is smaller than a cumulative average value of the respiratory cycles (‘reference cycle’) of the patient (S340).

As a result, when it is determined that the current respiratory cycle measured from the patient is smaller than the cumulative average value of the respiratory cycles (reference cycle), although there is no abnormality in the respiratory volume of the patient, the controller determines that the respiration becomes faster due to the sputum or the like, and transmits operation start commands to the suction pump 210 and the driving unit 230. Thereby, the catheter 100 starts to be inserted into the respiratory organ of the patient (S390).

Meanwhile, in the above-described step S340, if it is determined by the controller 270 that the current respiratory cycle measured from the patient is not shorter than the cumulative average value of the respiratory cycles (reference cycle), the controller 270 analyzes waveforms of the auscultatory sounds of the patient, and determines whether a maximum amplitude of the analyzed waveforms exceeds a predetermined reference amplitude value (S360).

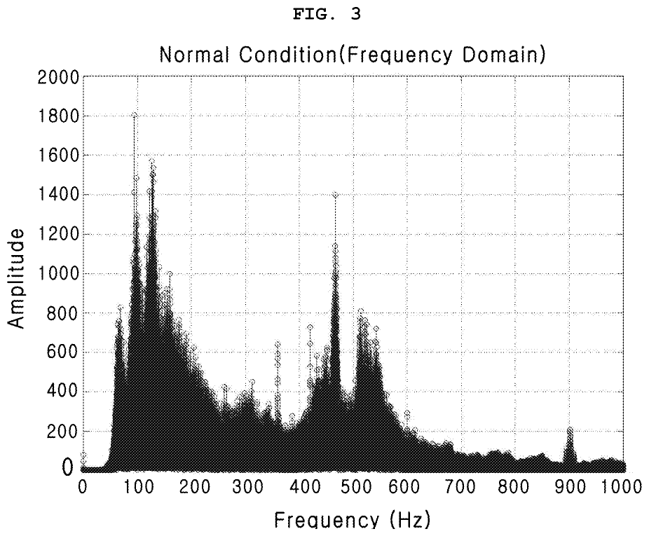

In particular, the controller 270 normally receives the auscultatory sounds (or the sound of breathing) from the patient's chest measured from the stethoscope microphone 240 in real time, and performs an analysis of the waveforms as illustrated in FIG. 3 for the received auscultatory sounds. FIG. 3 is a graph illustrating the waveforms of auscultatory sounds measured from the patient without respiratory disturbance caused by the sputum or the like with being converted into a frequency domain.

The controller 270 sets an average value obtained by accumulating the maximum amplitudes of the analyzed waveforms for the auscultatory sounds received from the stethoscope microphone in real time as a reference amplitude value.

Meanwhile, the controller 270 analyzes the auscultatory sounds received from the stethoscope microphone 240. At this time, if it is determined that the maximum amplitude of the analyzed waveforms exceeds the predetermined reference amplitude value, the controller 270 determines that the patient's breathing is irregular due to the sputum or the like, and transmits operation start commands to the suction pump 210 and the driving unit 230. Thereby, the catheter 100 starts to be inserted into the respiratory organ of the patient (S390).

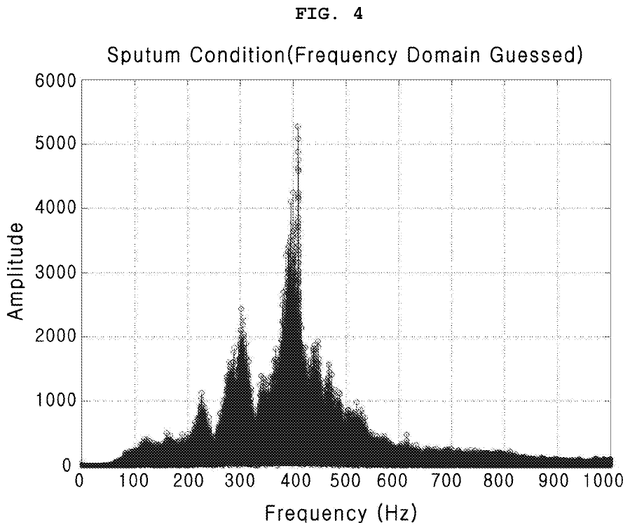

Specifically, as illustrated in FIG. 4, it can be seen that the maximum amplitude of the waveforms of the auscultatory sounds measured from the patient having respiratory disturbance due to the sputum or the like exceeds the maximum amplitude of the waveforms in a normal state from FIG. 3.

Meanwhile, in the above-described step S360, if it is determined by the controller 270 that the maximum amplitude of the waveforms analyzed for the auscultatory sounds received from the stethoscope microphone 240 does not exceed the predetermined reference amplitude value, the controller 270 determines whether the current pulse rate of the patient exceeds a reference pulse rate (S370).

Specifically, the controller 270 normally receives the pulse rates of the patient in real time from the pulse measurement unit 260 mounted on the patient's wrist, and sets an average value of the received cumulative pulse rates as the reference pulse rate.

Meanwhile, if it is determined by the controller 270 that the pulse rate received from the pulse measurement unit 260 exceeds the reference pulse rate, the controller 270 determines that an unconscious patient has difficulty in breathing due to the foreign matters such as sputum in the respiratory organ, consequently the pulse rate of the patient is increased, and transmits operation start commands to the suction pump 210 and the driving unit 230. Thereby, the catheter 100 starts to be inserted into the respiratory organ of the patient (S390).

Meanwhile, in the above-described step S370, if it is determined by the controller 270 that the pulse rate received from the pulse measurement unit 260 does not exceed the reference pulse rate, the controller 270 determines whether the current oxygen saturation measured from the patient is less than a reference oxygen saturation which corresponds to about 80% of the oxygen saturation in the normal state (S380).

Meanwhile, when it is determined by the controller 270 that the current oxygen saturation of the patient received from the oxygen saturation measurement unit 280 is less than the reference oxygen saturation, the controller 270 determines that the unconscious patient has difficulty in breathing due to the foreign matters such as sputum in the respiratory organ, consequently the oxygen saturation of the patient is decreased, and transmits operation start commands to the suction pump 210 and the driving unit 230. Thereby, the catheter 100 starts to be inserted into the respiratory organ of the patient (S390).

Meanwhile, when starting an insertion of the catheter into the respiratory organ after determining that the foreign matters such as sputum exist in the respiratory organ of the patient by the above-described method, the foreign matters may be successfully removed through the catheter. However, due to a misalignment in an insertion direction of the catheter into the respiratory organ, a situation, in which the suction end portion of the catheter is adsorbed to an inner wall of bronchial tubes, may occur.

In this case, the patient feels severe pain. Therefore, the inventor of the present invention has conceived a method of controlling an artificial intelligence medical suction device capable of intelligently determining whether the catheter is adsorbed to the inner wall of the bronchial tubes, and has completed the present invention based on the method.

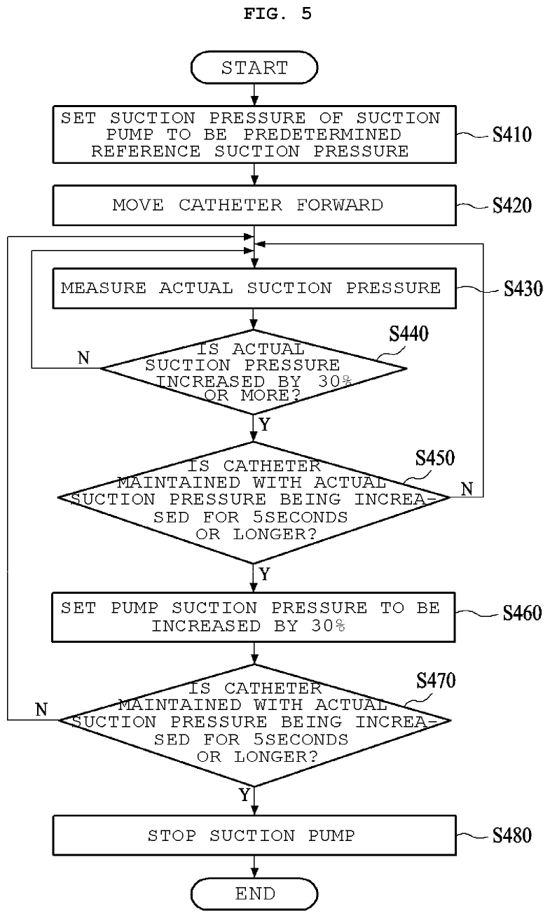

FIG. 5 is a flowchart illustrating a procedure and steps for executing the method of controlling the artificial intelligence medical suction device 200 for determining whether the catheter 100 is adsorbed to the inner wall of the bronchial tubes according to another embodiment of the present invention. Hereinafter, the steps for executing the method of controlling the artificial intelligence medical suction device 200 for determining whether the catheter 100 is adsorbed to the inner wall of the bronchial tubes according to another embodiment of the present invention will be described with reference to FIGS. 1 and 5.

First, the controller 270, which has determined to insert the catheter 100 into the respiratory organ, sets so that the suction pressure of the suction pump 210 is a predetermined reference suction pressure (S410), and then controls the driving unit 230 to move the catheter 100 forward (S420).

As described above, the suction pressure of the suction pump 210 is maintained at a constant value while the catheter 100 is inserted into the respiratory organ to move forward, and the pressure sensor 250 installed in the catheter 100 measures an actual suction pressure value inside the catheter 100 (S430).

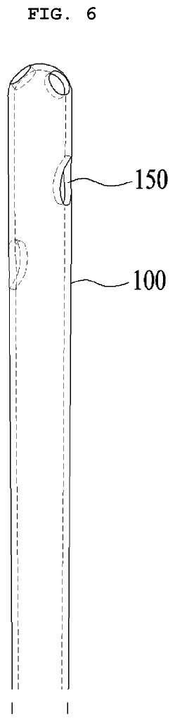

Specifically, as illustrated in FIG. 6, the catheter 100 has a plurality of suction holes 150 formed in the suction end portion thereof. If a part of the plurality of suction holes 150 is blocked due to contacting with the foreign matters such as sputum, the actual suction pressure value inside the catheter 100 is momentarily increased. Thereafter, when the foreign matters such as sputum are sucked through the suction hole 150 and discharged to an outside, the actual suction pressure value inside the catheter 100 is returned to the original state again.

However, when the foreign matters such as sputum, which are in contact with the suction hole 150 formed in the end portion of the catheter 100, have a high viscosity, the foreign matters cannot pass through the suction hole 150 even after a predetermined time elapses. Therefore, the suction hole 150 is maintained with being still blocked by the foreign matters.

Accordingly, in the present invention, when it is determined that the actual suction pressure value inside the catheter 100 is increased by a predetermined ratio (for example, 30%) or more (S440), the controller 270 determines that the catheter is maintained in such a pressure state for a predetermined reference time (for example, 5 seconds) or longer (S450).

Meanwhile, when the actual suction pressure value inside the catheter 100 is returned to the original state within a predetermined reference time, the controller 270 determines that the foreign matters which temporarily blocked the suction hole 150 are completely sucked into the catheter 100.

However, when the catheter is maintained with the actual suction pressure value therein being increased even if the predetermined reference time elapses, the controller 270 determines that the foreign matters have not passed through the suction hole 150 due to the high viscosity of the foreign matters blocking the suction hole 150, and increases the suction pressure value of the suction pump 210 by a predetermined ratio (for example, 30%) so that the foreign matters can be sucked (S460).

As described above, when the foreign matters pass through the suction hole 150 by the increased suction pressure of the suction pump 210, the actual suction pressure value inside the catheter 100 is returned to the value measured in the above-described step S430.

However, if the end portion of the catheter 100 is adsorbed to the inner wall of the bronchial tubes rather than the suction hole 150 is blocked by the foreign matters such as sputum, in spite of setting the pump suction pressure to be increased in the above-described step S460, the catheter 100 is maintained with the actual suction pressure value therein being still increased.

Accordingly, even after increasing the pump suction pressure in the above-described step S460, when the catheter 100 is maintained with the actual suction pressure value therein being increased for a predetermined reference time (for example, 5 seconds) or longer, the controller 270 of the present invention determines that the suction hole 150 is blocked due to the end portion of the catheter 100 is absorbed to the inner wall of the bronchial tubes, and stops the driving suction pump 210 to prevent the patient from suffering (S480).

Meanwhile, to embody the present invention, in the above-described step S450, when the actual suction pressure value inside of the catheter 100 is increased by a predetermined ratio or more, and the catheter is maintained in such a pressure state for a predetermined reference time (for example, 5 seconds) or longer, the controller 270 may determine that the suction hole 150 is blocked due to the end portion of the catheter 100 is absorbed to the inner wall of the bronchial tubes, and stops the driving suction pump 210 to prevent the patient from suffering (S480).

Terms used in the present application are used only to describe specific embodiments, and are not intended to limit the present invention. Singular expressions used herein include plural expressions unless they have definitely opposite meanings in the context. In the present application, it should be understood that term “include” or “have” indicates that a feature, a number, a step, an operation, a component, a part or the combination thereof described in the specification is present, but does not exclude a possibility of presence or addition of one or more other features, numbers, steps, operations, components, parts or combinations thereof, in advance.

While the present invention has been described with reference to the preferred embodiments and modified examples, the present invention is not limited to the above-described specific embodiments and the modified examples, and it will be understood by those skilled in the related art that various modifications and variations may be made therein without departing from the scope of the present invention as defined by the appended claims, as well as these modifications and variations should not be understood separately from the technical spirit and prospect of the present invention.

### INDUSTRIAL APPLICABILITY

The present invention can be applied to the medical suction device, such that industrial applicability thereof may be recognized in the medical device industrial fields.

1. A method of controlling an artificial intelligence medical suction device which removes foreign matters in a respiratory organ using a catheter, the method of controlling an artificial intelligence medical suction device comprising:
(a) setting, by a controller, a suction pressure of a suction pump connected to the catheter to be a predetermined reference suction pressure;
(b) moving, by the controller, the catheter forward so that a suction end portion of the catheter is inserted into the respiratory organ; and
(c) analyzing, by the controller, a state of the suction end portion of the catheter based on a variation value of an actual suction pressure measured inside of the catheter.

2. The method of controlling an artificial intelligence medical suction device according to claim 1, wherein, in step (c), when the catheter is maintained with the actual suction pressure measured therein being increased by a predetermined ratio or more for a predetermined time or longer, the controller determines that the suction end portion of the catheter is adsorbed to an inner wall of bronchial tubes.

3. An artificial intelligence medical suction device which removes foreign matters from a respiratory organ using a catheter, the artificial intelligence medical suction device comprising:
a suction pump connected to the catheter; and
a controller configured to set a suction pressure of the suction pump to be a predetermined reference suction pressure, move the catheter forward so that a suction end of the catheter is inserted into a respiratory organ, and analyze a state of a suction end portion of the catheter based on a variation value of an actual suction pressure measured inside of the catheter,
wherein, when the catheter is maintained with the actual suction pressure measured therefrom being increased by a predetermined ratio or more for a predetermined time or longer, the controller determines that the suction end portion of the catheter is adsorbed to an inner wall of bronchial tubes.
